SEQUENCE LISTING (3 sequences)

SEQ ID NO: 1 - DNA, 22 bp, artificial sequence
  primer
gtcccctacggacaaggcgcgt

SEQ ID NO: 2 - DNA, 27 bp, artificial sequence
  primer
tttttcgcgccttgtccgtaggggact

SEQ ID NO: 3 - DNA, 27 bp, artificial sequence
  primer
tttttcgcgccttgtccgtaggggact

(19) United States
(12) Patent Application Publication
(10) Pub. No.: US 2005/0112617 A1
(43) Pub. Date: May 26, 2005

(54) Method and device for the quantitative electrical detection of analytes

(75) Inventors: Edgar Diessel, Koln (DE); Jens Burmeister, Koln (DE)

Correspondence Address: NORRIS, MCLAUGHLIN & MARCUS, PA, 875 THIRD STREET, 18TH FLOOR, NEW YORK, NY, 10022, US

(73) Assignee: Bayer Technology Services GmbH, Leverkusen (DE)

(21) Appl. No.: 10/870,190

(22) Filed: Jun. 16, 2004

(30) Foreign Application Priority Data
Jun. 20, 2003 (DE) 103 27 683.1

(51) Int. Cl. C12Q 1/68; G01N 33/53

(52) U.S. Cl. 435/6; 435/7.1; 205/777.5

(57) ABSTRACT

The invention relates to a method for detecting one or more analytes by a recognition reaction, using a device with an electrolyte-filled electrolyte space, having at least one measurement electrode, optionally with an insulating region (insulator layer) next to the measurement electrode, and recognition molecules which are immobilized on the measurement electrode and/or on the insulating region next to the measurement electrode, and a counterelectrode. The electrical conductance or the resistance between the measurement electrode/s and the counterelectrode is measured as a function of the development time, and the time profile of the conductance or the resistance is analyzed. The invention also relates to a device with which the method according to the invention can be carried out.

The invention relates to a method and a device for the quantitative electrical detection of analytes in sample. This advantageously involves the specific detection of a biologically relevant molecule in an aqueous medium. Such a sensor principle, or such a sensor, has a wide range of application, for example in environmental analysis, the food industry, human and veterinary diagnosis, crop protection and in biochemical or pharmacological research.

For such diagnostic applications, bio- and chemosensors which have a biofunctional surface and a physical signal transducer are known. A biofunctional surface in the context of this invention is to be understood as a surface, on which biological, chemical or biochemical recognition elements are bound.

Biological, chemical or biochemical recognition elements, for example, DNA, RNA, aptamers, and receptors, to which an analyte binds specifically by means of a recognition reaction during detection, are bound to biofunctional surfaces.

Examples of recognition reactions are the binding of ligands to complexes, the sequestration of ions, the binding of ligands to (biological) receptors, membrane receptors or ion channels, of antigens or haptens to antibodies (immunoassays), of substrates to enzymes, of DNA or RNA to particular proteins, of aptamers or spiegelmers to their targets, the hybridization of DNA/RNA/PNA or other nucleic acid analogues (DNA assays), or the processing of substrates by enzymes.

Examples of analytes to be detected are DNA, RNA, PNA, nucleic acid analogues, enzyme substrates, peptides, proteins, potential active agents, medicaments, cells, and viruses.

Examples of recognition elements, to which the analytes to be detected bind, are DNA, RNA, PNA, nucleic acid analogues, aptamers, spiegelmers, peptides, proteins, sequestrants for metals/metal ions, cyclodextrins, crown ethers, antibodies or fragments thereof, anticalins, enzymes, receptors, membrane receptors, ion channels, cell adhesion proteins, gangliosides, and mono- or oligosaccharides.

If a variety of recognition elements are bound to the surface of the signal transducer so that they are spatially separated from one another, then a large number of recognition reactions can be carried out simultaneously with a sample to be studied. This is done, for example, with so-called DNA arrays in which various DNA sequences (for example oligonucleotides or cDNAs) are immobilized on a solid support (for example glass). Such DNA arrays are generally read by using optical methods, or alternatively by using electrical methods, and they are employed in expression profiling, sequencing, detection of viral or bacterial nucleic acids, or genotyping.

The recognition reaction in bio- or chemosensors may be detected by using optical, electrical or electrochemical, mechanical and magnetic signal transduction methods.

Although the most advanced described optical methods, in particular, have high sensitivities, they can generally be miniaturized only to a limited extent because of the complex structure involving a light source, sensor and photodetector, and they are therefore inferior to electrical methods with respect to production costs.

For these reasons, increased importance is being attached to the development of electrical sensors. In particular, the use of microstructuring techniques from semiconductor technology leads to miniaturized formats which offer high sensitivities with low production costs. In particular, methods which use labelling units for the analytes, the properties of which differ significantly from those of the constituents of the sample to be analyzed, are advantageous. To that end, for example, metal nanoparticles are suitable as labelling units.

In the scope of DC measurements, certain electrical biosensors with metal nanoparticles have the potential for extraordinarily high sensitivity, down to the single-molecule range. This potential is facilitated, in particular, by autometallographic deposition. In this so-called autometallography process, which is known from photography and electron microscopy, the nanoparticles or colloids act as catalysts for the electron transfer from a reducing agent to an Au or Ag ion, which the amplification solution contains in the form of an Ag or Au salt with the reducing agent, for example, hydroquinone. After reaction has taken place, the ion precipitates as metal onto the colloid. Electrode pairs, which are separated from one another by an insulator, are to that end selected as the electrical signal transducer. During autometallographic amplification, analyte molecules labelled with nanoparticles form a conductive bridge between the electrodes, and this is detected by a DC resistance measurement. The fundamental technique for this is described, for example, in: U.S. Pat. No. 5,284,748. Further disclosures relating to use for DNA selection can be found in WO 99/57550-A2 and in WO 01/00876-A2. The detection of nucleic acid by DC resistance measurement has been demonstrated (cf. Möller et al., Langmuir 17, 5426 (2001)). As a further development stage of this method, the discrimination of point mutations (single nucleotide polymorphisms (SNPs)) is described in (Park et al., Science 295, 1503 (2002)). Although quantification is possible using this method, this is very expensive since it is necessary to perform a chain of amplification cycles with subsequent point measurements.

Disadvantages of the known methods are complicated sequences of application, development, washing and drying steps, and the development, washing and drying steps may need to be repeated.

A practicable solution concept for quantification is described in WO 02/02810-A2. In this case, the electrically conductive deposit on the labelling units is detected during the deposition reaction by means of conductance measurements between two microelectrodes arranged in pairs. The conductance between the electrodes is modified by the formation of a metallic bridge as a result of the recognition reaction.

A disadvantage of this method is that these requirements necessitate two microstructure electrodes per measurement area, which are next to each other, so that perturbations of the conductance measurement due to accidental short circuits can therefore vitiate the measurement on DNA.

As an alternative to the formation of a metallic bridge between two electrodes, methods which use autometallographically amplified metal colloids to detect analytes bound on an electrode are known (Cai et al., Analytica Chimica Acta 469, 165-172 (2002)).

A disadvantage of this method is the comparatively small measurement area, which leads to a poor signal-to-noise ratio. Furthermore, possibly vitiating elaborate washing and drying steps are again needed in order to prepare for the measurement.

Likewise, the time profile of the growth process cannot be tracked by means of this.

It was an object of the invention to develop a highly sensitive electrical measurement device and a measurement method for the detection of analytes by means of recognition reactions, which avoids the disadvantages of the prior art.

The solution of the object according to the invention involves the following:

A method for detecting one or more analytes by a recognition reaction, using:
a device with an electrolyte-filled electrolyte space, comprising:
   at least one measurement electrode, optionally with an insulating region (insulator layer) next to the measurement electrode,
   recognition molecules which are immobilized on the measurement electrode and/or on the insulating region next to the measurement electrode/s, and a counterelectrode,
A) bringing analytes labelled with electrically active labelling units in contact with the recognition molecules, the electrically active labelling units either having been bound to the analytes before contact of the analytes with the recognition molecules or being bound to the analytes after contact of the analytes with the biofunctional surface,
B) applying a reactive amplification solution into the electrolyte space in order to amplify/produce the electrical conductance of the electrically active labelling units,
C) measuring the electrical conductance or the resistance between the measurement electrode/s and the counterelectrode as a function of the development time,
D) analysing the time profile of the conductance or the resistance according to C).

The invention in this embodiment relates to online measurement of the electrical resistance or the conductance between the measurement electrode and the counterelectrode of the measurement device while there is a suitable amplification solution on the measurement device. The measurement electrode, together with the insulating substrate region next to it, will be referred to below as a measurement area. The size of the measurement area is defined by the area of the associated region of the immobilized recognition molecules. It has been found that a significant change in the resistance or the conductance between the measurement electrode and the counterelectrode can be measured during the amplification process. One interpretation of the drop in resistance is that the conductive labelling units which are on the insulator then form a conductive layer and become conductively linked to the measurement electrode. The electrode area is greatly increased by this. The measurement electrode may be occupied by recognition DNA in this case. However, this is unnecessary if the labelling units become electrically conductively linked to the electrode during the formation of the conductive layer, owing to the spatial proximity to the measurement electrode of the labelling units on the insulator.

In order to increase the electrode area beyond the region of the immobilized recognition molecules by the amplification process, in a preferred embodiment of the sensor device, a conductive layer which is insulated from the measurement electrode and has a certain area is also applied in proximity to the measurement electrode. This area only needs to be partially occupied by the region of the sample molecules. Owing to the recognition reaction with subsequent amplification, this area becomes electrically conductively linked to the measurement electrode so that the conductance increase or resistance decrease is further enhanced.

All methods known from electrochemistry may be used for the detection, for example cyclo-voltammetry. In this context, potential circuits may be employed in a 3-electrode arrangement. To that end, in a preferred embodiment, a reference electrode is integrated into the measurement setup in addition to the measurement and counterelectrodes, the reference electrode being kept at a constant voltage relative to the measurement electrode. For reasons of simplicity, measurement in a particularly preferred embodiment is carried out only in a 2-point geometry with a measurement electrode and a counterelectrode. To that end, either a) a voltage or b) a current is applied between the measurement electrode or electrodes and a counterelectrode during the amplification process, and either in case a) the current or in case b) the voltage between the electrodes is measured during the amplification process. In general, the terms resistance and conductance measurements in the text cover the aforementioned methods both in a 2-point and in a 3-point geometry.

In particular, the online measurement allows quantification by analysis of the time profile of the resistance or the conductance. The greater the density of the electrically active labelling units on the measurement area after a recognition reaction has taken place, the earlier a significant resistance drop or conductance increase will be reached. This is reflected, in particular, by an extremum in the first derivative, i.e. the gradient, in the time profile. A monotonic relationship is found between the time taken to reach this situation and the concentration of the analyte, and this can be used for the quantification. In order to increase the accuracy of the quantification, the shape of the time profile curve of the electrical characteristic may be matched using a suitable mathematical function. In this case, the analyte concentration will be determined from the curve parameters. In order to quantify an analyte, the associated times or parameters from the curve match are compared with the analyte concentration known from at least one control sample.

Another preferred method is therefore characterized in that the concentration reading in a quantitative assay, or the basic detection in a qualitative assay, as a function of the reaction time B) until a significant gradient is reached in the time profile of the resistance or conductance, is compared with the reaction time B) of a reference sample, and is used for quantitative or qualitative analysis of the analyte.

The recognition molecules are, in particular, immobilized on the measurement area, and/or on the insulator layer next to it, by methods which are well known to the person skilled in the art. For DNA recognition units, this immobilization is described, for example in S. L. Beaucage, Curr. Med. 2001, 8, 1213-1244. For the immobilization on the measurement area, it is desirable to have an optimum density of recognition units which, with a high surface density, ensures optimum activity of the recognition unit. The recognition elements, such as antibodies, may be immobilized covalently or non-covalently. For example, avidin or streptavidin may be physisorbed onto the surface or covalently immobilized after suitable biofunctionalization of the surface. Biotinylated antibodies, for example, can be specifically immobilized onto the surface when it has been coated with avidin or streptavidin.

Recognition elements for the analytes are preferably bound to a measurement area with a biofunctional surface. The analytes enter into a recognition reaction with the recognition elements. The analyte may already be labelled with an electrically active labelling unit before the binding to the recognition element, or alternatively it is not labelled until after the binding to the recognition element, for example as a result of a binding element, which is labelled with a labelling unit, becoming bound to the complex consisting of the recognition element and the molecule.

The analytes may also be detected indirectly by the recognition reaction, and therefore need not necessarily be labelled. In the case of indirect detection, analytes which are already labelled with labelling units before binding to the recognition element are brought in contact with the biofunctional surface. At the same time, unlabelled analytes are also brought in contact with the biofunctional surface. These two species compete for binding to the immobilized recognition elements. If there are no unlabelled analytes in the electrolyte over the measurement area, then all the binding sites on the recognition elements will be occupied by labelled analytes, and the modification of the resistance or the conductance will be a maximum. In the event of a non-zero concentration of unlabelled analytes, some of the binding sites on the recognition elements will be occupied by unlabelled analytes, and some will be occupied by labelled analytes, according to the concentrations in question, so that the modification of the impedance is smaller compared to when the concentration of the unlabelled analyte is zero. This mixed system is calibrated by measuring at least one sample, which contains only the marked analyte at a known concentration.

A preferred method is therefore characterized in that a quantitatively predetermined amount of a known analyte, which is provided with labelling units, is mixed with the sample of an unlabelled known analyte in step A), and the concentration of the unlabelled analyte is determined from a comparison of the analysis D) of this mixed system with the analysis of the pure known labelled analyte.

With the method according to the invention, it is possible to detect the modification of the conductance or the resistance between the electrodes due to a single labelling unit per analyte molecule. Analyte molecules may furthermore be provided with a plurality of electrical-active labelling units, in order to increase the sensitivity of the method even further.

According to the preferred method, analytes are labelled with labelling units which are suitably active electrically. The electrical activity may consist in the electrical conductivity of the material used for the labelling units, which is preferably in the range of metallic conductivities. Monomers of electrically conductive polymers may furthermore be selected as electrically active elements. Lastly, enzymes are also considered as electrically active elements if they catalyze a reaction which leads to conductive products, for example conductive polymers.

Nanoparticles, metal complexes and/or clusters of conductive materials such as Au, Ag, Pt, Pd, and Cu may be used as electrically active labelling units.

The size of the electrically active labelling units is preferably in the range of from 1 to 100 nm, in particular preferably in the range of from 1 to 30 nm, and particularly preferably in the range from 1 to 2 nm. The latter size is produced, for example, by Au clusters consisting of 50-150 atoms. The indicated size refers in this case to the largest diameter of the labelling units.

Nonconductive particles with a conductive coating or nonconductive particles with a metallic coating may furthermore be used as labelling units. The nonconductive particles may, for example, be polystyrene beads.

Labelling units may preferably be based on conductive polymers such as polyanilines, polythiophenes, especially polyethylene dioxythiophene, polyphenylenes, polyphenylene vinylene, polythiophene vinylene, and polypyrrols.

Another preferred method is characterized in that the electrically active labelling units are based on enzymes, preferably HRP, which form electrically active labelling units by the reaction of a substrate selected from aniline or ethylene dioxythiophene.

A further use of HRP is the deposition of a polymer to which, for example, nanoparticles or all the labelling units described above are bound directly or indirectly via biotin-streptavidin, biotin-avidin or biotin-NeutrAvidin. For the indirect case, the polymer is biotinylated. This principle is referred to as catalyzed reporter deposition (CARD).

A particularly preferred method is characterized in that the electronically active labelling units are based on an enzyme which catalyzes the formation of a nonconductive polymer, in particular a biotinylated polymer, which is in turn directly or indirectly connected to nanoparticles, metal complexes or clusters based on elements from the list: Au, Ag, Pt, Pd, and Cu, or to electrically conductive polymers.

Suitable amplification solutions are dependent on the nature of the selected electrically active labelling units. Autometallographic amplification solutions based on Ag or Au salts are particularly advantageously used for the signal amplification in the case of nanoparticles, metal complexes and/or clusters of conductive materials, nonconductive particles with a metallic coating. Hydroquinone or formaldehyde, for example, are used as a reducing agent in this case. Amplification solutions for monomers of electrically conductive polymers may consist of catalysts, initiators and/or other monomers of these polymers, which are needed for the polymerization. For example, HRP may be used as a catalyst for the polymerization of aniline. The monomers of an electrically conducting polymer, for example aniline, may be used as the amplification solution for enzymes as electrically active labelling units, for example HRP.

Amplification solutions may change their concentration above electrically conductive labelling units in the course of the amplification process, so that the amplification process enters a saturation phase. The amplification solution should preferably be replaced in order for the amplification process to continue in this case. This may be done by stirring or complete replacement of the liquid, for example in the scope of a microfluidic flow system. The replacement is preferably carried out continuously.

The method according to the invention may, for example, be used for the analysis of peptides, proteins or nucleic acids. The recognition reaction A) used in the invention is preferably a peptide or protein assay, in particular an immunoassay or a nucleic acid assay, in particular an RNA or DNA assay, preferably an SNP assay. DNA assays are preferably used for detecting viral DNA or RNA, or DNA of bacterial species, as well as expression profiling, genotyping for the diagnosis of hereditary diseases or for pharmacogenomics (genetically related activity or side-effects of pharmaceuticals), nutrigenomics (general related activity or side-effects of foodstuffs). In particular, modifications of genes which are due to the variation of only one base (single nucleotide polymorphism=SNP) are established in genotyping.

The method according to the invention allows the simultaneous analysis of a multiplicity of analytes by providing a corresponding multiplicity of measurement areas for such multiplex analyses. In this case, one analyte is detected per measurement area. The same recognition element may also be immobilized on each of a plurality of measurement areas for multiple detection of one analyte. Individual measurement areas may, for example, be used to detect reference substances which characterize the effect of, for example, temperature, or light on the amplification process. These reference values can be used to normalize the signals from other measurement areas. Another preferred method is therefore characterized in that a multiplicity of recognition reactions with the steps A) to D) are simultaneously carried out in parallel on a sensor device by providing the measurement device with a multiplicity of measurement areas, on each of which the same or different recognition elements are applied. Multiplex assays with numbers >1000 are employed for scientific applications, whereas numbers of 1-1000 are appropriate for diagnostic applications. Owing to the possible multiplicity of measurement areas, the device can simultaneously be used as a protein array or peptide array to detect a multiplicity of proteins or peptides, or as a nucleic acid array to detect nucleic acids. In particular, up to 1000 recognition reactions may be carried out simultaneously on one sensor device.

The detection of the analyte is, in particular, carried out in bodily fluids such as blood, saliva, urine, sweat, interstitial fluid and tear fluid.

The invention also relates to a device for detecting one or more analytes by a recognition reaction, in particular using the described method, at least comprising:
at least one measurement electrode and at least one adjacent insulating region (insulator layer),
recognition molecules, which are immobilized on the insulating region next to the electrode and optionally on the electrode,
a counterelectrode,
analytes, which are labelled with electrically active labelling units
optionally an amplification solution, and
a measuring instrument for time-resolved recording of the conductance or the resistance between the measurement electrode and the counterelectrode during the amplification process.

In the device according to the invention, the measurement areas, recognition elements, analytes, electrically active labelling units and amplification solutions preferably have the properties described above for carrying out the method.

The conductance is, for example, measured in a 2- or 3-point geometry. In both arrangements, the counterelectrode or the reference and counterelectrode together with the measurement electrode may be accommodated on a common substrate or designed as separate electrodes in a measurement cell. This allows cost-efficient production of the measurement cell.

According to the method, one or preferably a plurality of measurement areas are applied to the substrate. Accordingly, one species of recognition elements is immobilized on each of the individual measurement areas. Different measurement areas may respectively carry the same or pairwise different species of recognition elements.

A preferred device is therefore characterized in that the surface of the measurement device has a multiplicity of measurement areas, on each of which the same or different recognition elements are applied.

The measurement electrode may advantageously be produced by screen printing techniques with structure widths of between about 100 μm and 1 mm. Optical lithography methods permit lateral structure sizes of about 2 μm. Substantially smaller lateral dimensions are achieved by electron-beam techniques. The smaller the measurement electrode is compared with the immobilized region of the recognition elements, the higher the achievable sensitivity is likely to be. With measurement areas of 100 μm², for example, it is possible to accommodate 10⁶ elements on a chip with a size of 100 mm². For diagnostic applications with about 100 analytes, conversely, measurement areas of up to 1 mm² can be produced on the same area with comparatively little outlay. These size indications are merely exemplary in nature, and do not preclude other sizes and numbers. Multiplex circuits are used in order to drive many electrodes.

The lateral width of the measurement area occupied by recognition elements is preferably from 100 μm to 1 mm.

Owing to the high possible packing density of the measurement areas, the device according to the invention is suitable as a platform for nucleic acid and protein arrays.

Another preferred embodiment of the device is characterized in that the sets of recognition elements with measurement electrode forms a nucleic acid array, a peptide array or a protein array.

In order to avoid depleting the amplification solution unspecifically by electrochemical processes at the electrical supply leads, the supply leads to the electrodes are preferably insulated from the amplification solution. This may, for example, be done by depositing SiO₂ on the supply leads.

The electrodes may be configured in a planar fashion or in a non-planar geometry.

Another preferred device is characterized in that the measurement areas are configured next to one another in a planar fashion on a substrate.

In the planar embodiment, there are one or more measurement areas laterally next to one another on a substrate. Analyte and amplification solutions can be delivered to the measurement areas via microchannels, which are etched into the substrate. In this case, for example, the measurement areas are at the bottom of these channels. As an alternative, a component provided with microchannels may be used as a cover for a planar substrate.

Particularly advantageously, a plurality of measurement areas may be configured vertically above one another in the form of alternating layer structures of electrodes and insulator layers, since deposition methods from semiconductor technology can be employed in this case. In addition, these layer stacks may be provided with microchannels, applied parallel to the surface normals of these layers, through which the analyte and amplification solutions are applied to the measurement areas. A multiplex assay is, in particular, carried out by applying a set of microchannels in a layer structure respectively with at least a cover layer and a base layer (electrical insulator) and at least one interlayer of electrically conducting layers (measurement layers) and insulator layers applied alternately next to one another. The channels are arranged next to one another and pass mutually independently through electrically driveable measurement layers, the individual channels being equipped with different recognition DNAs. As an alternative, a plurality of measurement areas lying above one another in a layer stack may be produced with the microchannels passing through a plurality of layers, vertically insulated from one another by insulator layers, of conducting measurement layers and insulator layers applied alternately next to one another. When electrical contact is made with the individual conducting measurement layers, pairs of electrodes and insulators arranged above one another are thus produced in individual microchannels. If different recognition DNAs are selectively immobilized between the electrodes of a single microchannel of the latter arrangement, a multiplexable microchannel for the online resistance or conductance measurement is produced with this structure. The spatially resolved DNA immobilization in a microchannel may be carried out by electrical attraction of polystyrene beads, on the surface of which recognition DNA is immobilized, between two preselectable electrodes, see Velev et al., Langmuir 15, 3693 (1999).

The sample delivery is preferably implemented via microchannels which, perpendicular to the measurement area, have a height in the range of between 1 and 1000 μm, preferably 1-50 μm. The indicated sizes are motivated by the diffusion coefficients of biomolecules in conjunction with incubation times in the second to lower minute range of 1-10 minutes.

A preferred alternative embodiment of the device is characterized in that the electrolyte space is formed by one or more channels, which have a multiplicity of measurement areas and have a height of at most 100 μm above the measurement electrodes.

In order to prevent depletion of the amplification solution induced by the electrically active labelling units, the amplification solution is preferably replaced gradually or continuously through the microfluidic channels described above. A device which drives the fluid replacement is furthermore to be provided. Pumps or syringes may be used to that end. Electro-osmotic fluxes may furthermore be produced by additional electrodes.

The nonconductive substrates preferably consist of a material selected from the list: glass, SiO₂, plastics, for example, polyethylene terephthalate, polycarbonate, or polystyrene.

Metals such as Au, Pl, Ag, and Ti, semiconductors such as Si, in particular doped Si, metal oxides, in particular indium-tin oxide (ITO), or conductive polymers such as polyanilines, polythiophenes, in particular polyethylene dioxythiophene, polyphenylenes, polyphenylene vinylene, polythiophene vinylene, or polypyrrols, are suitable for the electrodes and the conductive layers.

The invention also relates to the use of the device as a nucleic acid array, a peptide array or as a protein array.

The described method and device allow a simpler electrode structure in relation to the closest prior art (WO 02/02810), since metallic bridging of closely neighbouring electrodes is unnecessary and only one common counterelectrode is needed. In relation to the closest prior art (Cai et al., Analytica Chimica Acta 469, 165-172 (2002)), the described method leads to higher expected sensitivities owing to the formation of an electrically conductive layer on the measurement area. Owing to the measurement of the conductance taking place during the amplification, the amplification process can furthermore be assessed and stopped to save time if appropriate, and additional steps such as washing steps are also obviated.

### BRIEF DESCRIPTION OF THE DRAWING

The invention will be explained in more detail below by way of example with reference to the figures, in which:

FIG. 1 shows a diagram of the measurement device;

FIG. 2 shows a resistance-time diagram of a DNA hybridization with a control hybridization reaction;

FIG. 3 shows a sensor with an additional conductive area; and

FIG. 4 shows the vertical arrangement of the electrode/insulator layer sequences.

The following examples are intended to be illustrative of the present invention and not limiting in any way.

### EXAMPLES

### Example 1

Method and Device for Detecting DNA on a Polymer Chip

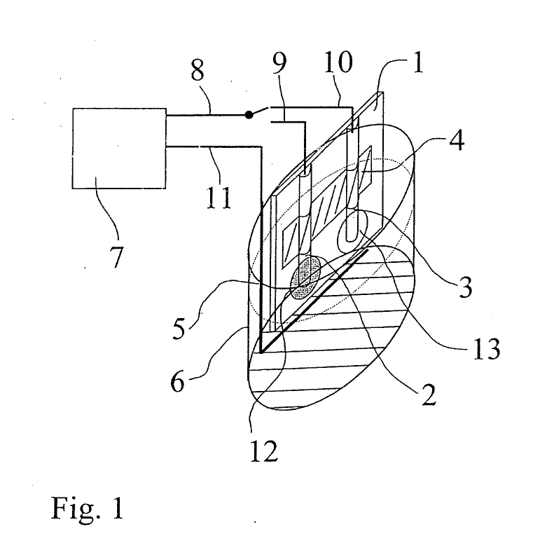

The sensor of the measurement device consists essentially of a polyethylene terephthalate substrate 1 with printed carbon electrodes 2 and 3, see FIG. 1. An insulating polymer layer 4, which lies between the terminal points of the sensor and the region of the measurement electrodes, is applied over the electrodes 2 and 3. The sensors were obtained from Molecular Circuitry, West Conshohocken, Pa., USA. An Ag wire 5, which is mounted about 1 mm away from the sensor in a Petri dish 6, is used as the counterelectrode. A Keithley 2000 multimeter, which is operated in the resistance mode, measurement range 10 Mohm, is used as the measuring instrument 7. The inputs of the measuring instrument can be selectively connected via the supply leads 8, 9, 10 to the electrodes 2 or 3 and to the silver wire 5 via the supply lead 11.

For purification, the sensors were placed in ethanol for 30 min in a first purification step, and subsequently rinsed with ddH₂O. This purification step was repeated once. In the second purification step, these sensors were placed in 2% strength Alkonox solution for 15 min and subsequently rinsed with ddH₂O. This purification step was also repeated once.

The sensors were incubated in an aqueous poly(phenylalanine)-lysine (Sigma) (0.1 mg/ml) solution with 2M NaCl and 50 mm KH₂PO₄ (pH 7.1) for 1 h at RT, in order to form a polymer layer on the sensor.

Two different regions of recognition elements were immobilized on the sensors: recognition DNA positive (5′-amino-GTCCCCTACGGACAAGGCGCGT-3′) (SEQ ID NO: 1) on the positive region 12 and recognition DNA negative (5′-amino-TTTTTCGCGCCTTGTCCGTAGGGGACT-3′) (SEQ ID NO: 2) on the negative region 13. This makes it possible to have positive and negative controls simultaneously in an assay while only providing a positive analyte.

Recognition DNAs were dissolved in phosphate buffer pH 7.2 and incubated with 0.1M bis-sulfo-succinimidyl suberate (BS3, manufacturer Pierce) for 10 min at RT. The reaction was terminated by dilution with phosphate buffer. The recognition DNA was purified by chromatography on an NAP 10 column (manufacturer Pharmacia). The purified recognition DNA was applied to the surface of the sensor in volumes of, for example, 20 μl and incubated overnight at room temperature. The resulting DNA chips were washed with 1% strength aqueous ammonium hydroxide solution and ddH₂O. The sensor was incubated for 4 h with an activated carboxymethyl dextran solution in order to block the unreacted amino groups on the chip surface. The solution was prepared as follows: part 1: 20 mg/ml carboxymethyl dextran in ddH₂O and part 2: 0.2 mmol/ml EDC+0.2 mmol/ml NHS in ddH₂O. Mix solution part 1+solution part 2 in the ratio 1:1 and react for 20 min. The sensors were subsequently incubated for 1 h in ddH₂O.

On the sensor surfaces 12 and 13 coated with recognition DNAs, DNA hybridization reactions were carried out with an analyte DNA sample having the sequence 5′-biotin-TTTTTCGCGCCTTGTCCGTAGGGGACT-3′ (SEQ ID NO: 3). A 10-8 M solution of the DNA in Tris buffer pH 8, 1M NaCl, 0.005% SDS was incubated on the sensor in a volume of 20 μl overnight at 56° C. Washing was subsequently carried out with hybridization buffer, in order to remove unhybridized DNA from the chip surface. The hybridized target DNAs were incubated for 4 h at RT with a solution of streptavidin-gold (diameter of the gold particles 10 nm, Sigma). The sensors were washed with hybridization buffer in which 1 M NaCl was replaced by 1 M NaNO₃, water and subsequently dried at RT. 50 μl of the amplification solution (1:1 mixture of (4.8 μl 1 M AgNO₃ to 0.2 ml 0.3 M citrate buffer) and (61 mg hydroquinone to 5 ml 0.3 M citrate buffer) were added to the sensor for 1 min. The sensors were subsequently rinsed 2× with ddH₂O and dried.

For the online measurement, the sensor was immersed in about 6 ml of the amplification solution (1:1 mixture of (4.8 μl 1 M AgNO₃ to 0.2 ml 0.3 M citrate buffer) and (61 mg hydroquinone to 5 ml 0.3 M citrate buffer).) At intervals of 20 s, the resistance measuring instrument 7 was alternately connected for about 10 s between the electrodes 9, 10 and the counterelectrode 11, and the resistance was measured.

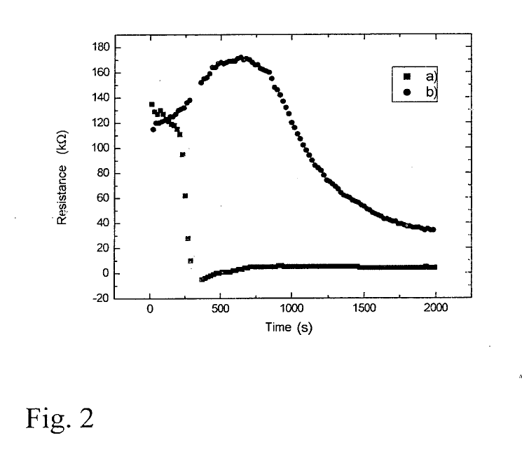

FIG. 2 shows the resistance as a function of time during the Ag amplification process for a) the positive reaction of the analyte DNA with the recognition DNA positive and b) the control hybridization reaction of analyte DNA with the recognition DNA negative. A significant abrupt reduction in the resistance is detected at t=220 s for the positive reaction. In comparison with this, the resistance drop for the negative reaction takes place significantly later and not so steeply.

### Example 2

Method and Device for Detecting DNA with an Additional Conductive Layer

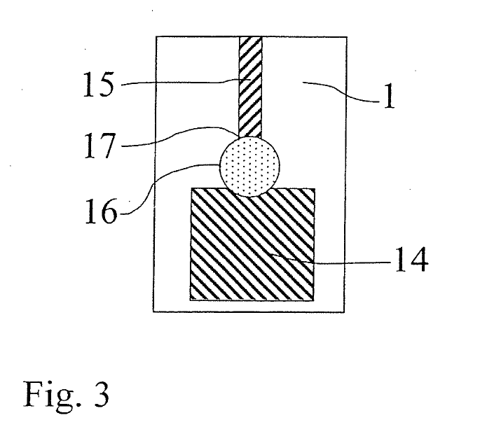

FIG. 3 represents another embodiment which permits binding, effected by the recognition reaction, of a conductive Au surface 14 to the carbon measurement electrode 15. The conductive surface and the electrode were applied to an insulating polyethylene terephthalate substrate 1 as in Example 1. The measurement area is defined by the immobilization region 16 and therefore covers both the end 17 of the electrode 15 defines and the end of the conductive Au surface 14. The preparation of this sensor, conduct of a hybridization reaction and the Ag amplification with simultaneous resistance measurement are carried out similarly as in Example 1.

### Example 3

Detection of DNA with a Device Having a Stacked Electrode/Insulator Sequence

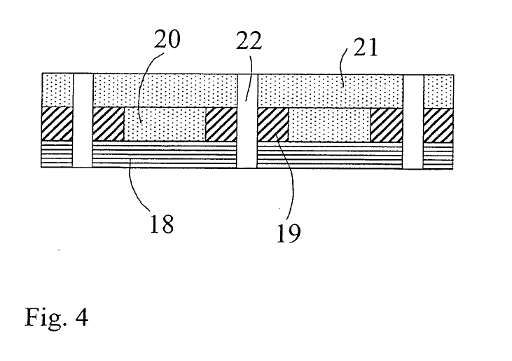

An alternative embodiment of a sensor according to the invention is a stacked electrode/insulator layer sequence according to FIG. 4. An Au layer is deposited on a glass substrate 18. This layer is divided into individual regions 19 by means of photolithography. The gaps 20 are subsequently filled with SiO₂ using a plasma deposition method. An SiO₂ cover layer 21 is applied to this layer by the same method. A microchannel 22 with a width of, for example, 20 μm, is formed through the layer structure by ion-beam etching. The immobilization of amino-functionalized DNA is achieved using known silanization methods, for example, with aminopropyltriethoxysilane (APTES) on the SiO₂ inside of this microchannel and a biofunctional BS³ linker from Example 1. In this example, different recognition DNAs are respectively immobilized on the measurement areas of the individual channels for the simultaneous detection of different analytes. The conduct of the assay and Ag amplification with simultaneous resistance measurement are carried out similarly as in Example 1, except that the reaction solutions are flushed through the openings of the device with a syringe-operated suction system. The electrical resistances of the individual regions 19 are successively or simultaneously measured against a single counterelectrode in the reaction vessel.

The foregoing is only a description of a nonlimiting number of embodiments of the present invention. It is intended that the scope of the present invention extend to the full scope of the appended issued claims and their equivalents.

1. Method for detecting one or more analytes by a recognition reaction, using: a device with an electrolyte-filled electrolyte space, comprising:
(i) at least one measurement electrode, optionally with an insulating region (insulator layer) next to the measurement electrode,
(ii) recognition molecules which are immobilized on the measurement electrode and/or on the insulating region next to the measurement electrode, and
(iii) a counterelectrode,
said method comprising the following steps:
A) bringing one or more analytes labelled with electrically active labelling units into contact with the recognition molecules, the electrically active labelling units either having been bound to the one or more analytes before contact of the one or more analytes with the recognition molecules or being bound to the one or more analytes after contact of the one or more analytes with a biofunctional surface,
B) applying a reactive amplification solution into the electrolyte space in order to amplify/produce the electrical conductance of the electrically active labelling units,
C) measuring the electrical conductance or the resistance between the measurement electrode/s and the counterelectrode as a function of the development time, and
D) analysing a time profile of the electrical conductance or the resistance measured in C).

2. Method according to claim 1, wherein a concentration reading in a quantitative assay, or a basic detection in a qualitative assay, as a function of a reaction time in step B) until a significant gradient is reached in a time profile of the resistance or conductance, is compared with a reaction time in step B) of a reference sample, and is used for quantitative or qualitative analysis of the analyte.

3. Method according to claim 1, wherein the recognition molecules are applied by means of a biofunctional surface coating on the measurement electrode and/or an adjacent insulator layer.

4. Method according to claim 1, wherein a quantitatively predetermined amount of a known analyte, which is provided with labelling units, is mixed with a sample of an unlabelled known analyte in step A), and the concentration of the unlabelled analyte is determined from a comparison of the analysis in step D) of this mixed system with an analysis of the pure known labelled analyte.

5. Method according to claim 1, wherein an analyte molecule is labelled with one or more electrically active labelling units.

6. Method according to claim 1, wherein the electrically active labelling units are nonconductive particles based on polymers with a conductive coating.

7. Method according to claim 1 wherein the electrically active labelling units are based on conductive polymers.

8. Method according to claim 1, wherein a multiplicity of recognition reactions with the steps A) to D) are simultaneously carried out in parallel on a sensor device by providing the device with a multiplicity of measurement areas, on each of which the same or different recognition elements are applied.

9. Device for detecting one or more analytes by a recognition reaction, at least comprising:
(a) at least one measurement electrode and at least one adjacent insulating region (insulator layer),
(b) recognition molecules, which are immobilized on the insulating region next to the electrode and optionally on the electrode,
(c) a counterelectrode,
(d) analytes, which are labelled with electrically active labelling units,
(e) optionally an amplification solution, and
(f) a measuring instrument for time-resolved recording of a conductance or a resistance between the measurement electrode and the counterelectrode during an amplification process.

10. Device according to claim 9, wherein the counterelectrode or the reference and counterelectrode together with the measurement electrode are accommodated on a common substrate.

11. A DNA array or a protein array comprising the device according to claim 9.
